SEQUENCE LISTING (14 sequences)

SEQ ID NO: 1 - protein, 5 aa, Artificial Sequence
  amyloid protein derived peptide
  SITE (1)..(1): May be modified with an N-terminal Fmoc moiety
VTAVA

SEQ ID NO: 2 - protein, 5 aa, Artificial Sequence
  amyloid protein derived peptide
  SITE (1)..(1): May be modified with an N-terminal Fmoc moiety
VTVVA

SEQ ID NO: 3 - protein, 5 aa, Artificial Sequence
  amyloid protein derived peptide
  SITE (1)..(1): May be modified with an N-terminal Fmoc moiety
VTAVV

SEQ ID NO: 4 - protein, 5 aa, Artificial Sequence
  amyloid protein derived peptide
  SITE (1)..(1): May be modified with an N-terminal Fmoc moiety
VLAVA

SEQ ID NO: 5 - protein, 5 aa, Artificial Sequence
  amyloid protein derived peptide
  SITE (1)..(1): May be modified with an N-terminal Fmoc moiety
VYAVA

SEQ ID NO: 6 - protein, 5 aa, Artificial Sequence
  amyloid protein derived peptide
  SITE (1)..(1): May be modified with an N-terminal Fmoc moiety
VRAVA

SEQ ID NO: 7 - protein, 5 aa, Artificial Sequence
  amyloid protein derived peptide
  SITE (1)..(1): May be modified with an N-terminal Fmoc moiety
VHAVV

SEQ ID NO: 8 - protein, 5 aa, Artificial Sequence
  amyloid protein derived peptide
  SITE (1)..(1): May be modified with an N-terminal Fmoc moiety
VHAVA

SEQ ID NO: 9 - protein, 5 aa, Artificial Sequence
  amyloid protein derived peptide
  SITE (1)..(1): May be modified with an N-terminal Fmoc moiety
VFAVA

SEQ ID NO: 10 - protein, 5 aa, Artificial Sequence
  amyloid protein derived peptide
  SITE (1)..(1): May be modified with an N-terminal Fmoc moiety
VWAVA

SEQ ID NO: 11 - protein, 5 aa, Artificial Sequence
  non-amyloid protein derived peptide
  SITE (1)..(1): May be modified with an N-terminal Fmoc moiety
LLFYL

SEQ ID NO: 12 - protein, 5 aa, Artificial Sequence
  non-amyloid protein derived peptide
  SITE (1)..(1): May be modified with an N-terminal Fmoc moiety
LLLYL

SEQ ID NO: 13 - protein, 5 aa, Artificial Sequence
  non-amyloid protein derived peptide
  SITE (1)..(1): May be modified with an N-terminal Fmoc moiety
LLVYL

SEQ ID NO: 14 - protein, 5 aa, Artificial Sequence
  non-amyloid protein derived peptide
  SITE (1)..(1): May be modified with an N-terminal Fmoc moiety
  SITE (3)..(3): Xaa can be any amino acid
LLXYL

(19) United States
(12) Patent Application Publication
(10) Pub. No.: US 2022/0088275 A1
(43) Pub. Date: Mar. 24, 2022

(54) HYDROGELS, METHODS OF MAKING, AND METHODS OF USE

(71) Applicant: Convalesce Inc., San Francisco, CA (US)

(72) Inventor: Subhadeep DAS, San Francisco, CA (US)

(21) Appl. No.: 17/612,882

(22) PCT Filed: May 20, 2020
(86) PCT No.: PCT/US2020/033869

§ 371 Date: Nov. 19, 2021

Related U.S. Application Data
(60) Provisional application No. 62/850,513, filed on May 20, 2019.

(51) Int. Cl. A61L 27/52; A61L 27/22; A61L 27/38; A61L 27/54; C12N 5/0775

(52) U.S. Cl. CPC A61L 27/52; A61L 27/227; A61L 27/3878; A61L 27/3834; A61L 27/3895; C12N 2533/52; C12N 5/0662; A61L 2300/414; C12N 2501/119; C12N 2501/41; A61L 27/54

(57) ABSTRACT

Provided herein are hydrogels comprised of self-aggregating peptides, useful for implantation into a subject, for the treatment of a neurological disease or disorder. The hydrogels may comprise growth factors and/or stem cells, e.g. neural stem cells. In various embodiments, the hydrogels aid stem cell viability, encourage growth and differentiation, and constrain implanted stem cells to a desired location within a subject. Also provided herein are methods of making and using the hydrogels.

### CROSS REFERENCE TO RELATED APPLICATIONS

This application claims priority to U.S. Provisional Application Ser. No. 62/850,513, filed May 20, 2019, which is herein incorporated by reference in its entirety for all purposes.

### DESCRIPTION OF TEXT FILE SUBMITTED ELECTRONICALLY

The present application is being filed with a Sequence Listing in electronic format. The Sequence Listing is provided as a file entitled COLE_001_01WO_SeqList_ST25.txt, created on May 20, 2020, and is 4.2 kilobytes in size. The contents of the text file submitted electronically herewith are incorporated herein by reference in their entirety.

### BACKGROUND

Neurological diseases may result from dysfunction or loss of neurons in the central nervous system. Neurodegenerative diseases such as Parkinson's disease and Alzheimer's disease, types of neurological diseases, involve the progressive degeneration of neurons in certain brain regions, resulting from a variety of mechanisms, many of which are not well understood. Current treatments for neurological diseases rely mostly on pharmacological and surgical approaches, most of which focus on suppressing symptoms rather than curing the underlying disease. For example, pharmacological treatment of Parkinson's disease involves delivery of dopamine or a dopamine precursor exogenously to alleviate motor symptoms. However, these treatments have limited efficacy in terms of the extent of symptomatic relief and often present severe side effects.

It has been hypothesized that stem cells can be implanted into the brain and encouraged to differentiate into functional neurons, replacing dead, damaged, or otherwise dysfunctional neurons in a diseased brain. However, problems with stem cell based therapies include the need to introduce large numbers of cells into the brain to achieve a sufficient number of differentiated cells to effectively replace the dead, damaged, or otherwise dysfunctional neurons. Usually, only a subset of the stem cells introduced into the brain eventually form into fully-differentiated neurons, while the rest often die; such dead cells can release cytokines resulting in a toxic environment. Conversely, introducing too low a number of cells in the brain is generally ineffective because too few cells differentiate to effectively treat the disease. Therefore, methods of treating neurological diseases that allow introduction of an optimal number of cells into the brain while still resulting in an effective amount of differentiated stem cells are desired. Further, in current therapeutic methods, transplanted stem cells often do not differentiate into the desired type of cell once implanted. Current methods lack a protocol for exposing stem cells to suitable differentiation cues prior to implantation. Therefore, a method of treating a disease that involves targeted differentiation in situ is highly desirable.

### SUMMARY

Described herein are compositions comprising hydrogels for use in treating disease, and methods of making the same. In some embodiments, hydrogels described herein for implantation into a subject comprise modified stem cells and/or one or more growth factors. In some embodiments, hydrogels may be generated from self-aggregating peptides derived from an amyloid protein. The stem cells may be modified prior to being loaded into the hydrogel. For example, the stem cells may be modified by culturing in a media comprising one or more growth factors. The stem cells may be modified to a partially differentiated state. In some embodiments, at least a subset of the self-aggregating peptides used to form the hydrogel are derived from an alpha-synuclein protein. In other embodiments, the self-aggregating peptides used to form the hydrogel may be derived from a protein that is not alpha-synuclein or beta amyloid. The peptides may comprise a modified non-amyloid-β component (NAC) region derived from an alpha-synuclein protein. In some embodiments, the modified NAC region does not comprise a tyrosine amino acid. The peptides may further comprise an Fmoc protected N-terminus. The amino acid sequence of the peptides may be selected from the sequences presented in Table 2. For example, the amino acid sequence of the peptides may comprise VHAVA (SEQ ID NO: 8). The peptides may have a length of approximately 2-12 amino acids. In some embodiments, the hydrogel may be generated from a plurality of identical self-aggregating peptides. In other embodiments, the hydrogel may be generated from a mixture of different self-aggregating peptides.

Stem cells may be cultured in media until a subset of cells reach a dopaminergic state. For example, stem cells may be cultured in media until a subset of cells exhibit upregulated tyrosine hydroxylase, nestin, and/or beta-III tubulin expression. In some embodiments, the media used to culture the cells does not comprise the growth factor FGF-2. In some embodiments, the media may comprise Sonic Hedgehog (SHH) and FGF-8 growth factors. The stem cells may be cultured in media for approximately 4-6 days. The stem cells in the hydrogel may be capable of differentiating to produce functional neurites. The stem cells in the hydrogel may be capable of differentiating into functional neurons. In some embodiments, the stem cells are mesenchymal stem cells. The hydrogel may comprise cells at a concentration within a range of approximately 200-4000 cells per μg of peptide. The hydrogel may have a pH in the range of 6.5-7.5. In some embodiments, the hydrogel may comprise NaCl. In other embodiments, the hydrogel may not comprise NaCl, or may comprise a negligible amount of NaCl. The pore size of the hydrogel may be between 2 and 15 μm. The hydrogel may be stable for 14-42 days when implanted into a subject. The modified stem cells within the hydrogel may remain viable for at least 15-20, 20-15, 25-30, 30-35, or 35-40 days after the hydrogel is implanted into a subject.

In some embodiments, hydrogels may be generated from self-aggregating peptides. The peptides may be derived from one or more non-amyloid proteins, wherein the one or more proteins comprise a region which, when isolated, forms β-sheet rich structures. In some embodiments, hydrogels may be generated from a plurality of identical self-aggregating peptides derived from non-amyloid proteins. In other embodiments, hydrogels may be generated from a mixture of different self-aggregating peptides derived from non-amyloid proteins. The protein from which the self-aggregating peptides are derived may be laminin. In some embodiments, peptides may comprise an Fmoc protected N-terminus. In some embodiments, the amino acid sequences of the peptides are selected from the sequences presented in Table 3. In some embodiments the amino acid sequences of the peptide comprises SEQ ID NO: 11. In some embodiments the amino acid sequences of the peptide comprises SEQ ID NO: 13 In some embodiments the amino acid sequences of the peptide comprises SEQ ID NO: 14. In some embodiments the hydrogel forms within 24 hours of peptide incubation. Hydrogels generated from peptides derived from one or more non-amyloid proteins may comprise one or more growth factors. Hydrogels may be loaded with stem cells. The stem cells may be modified stem cells. The stem cells may be mesenchymal stem cells. A subset of the cells may be in a dopaminergic state. In some embodiments, a subset of the cells may express nestin and/or beta-III tubulin. A subset of the cell may exhibit upregulated tyrosine hydroxylase expression. The hydrogel may comprise cells at a concentration within the range of approximately 200-4000 cells per μg of peptide. The hydrogel may have a pH in the range of 6.5-7.5. In some embodiments, the hydrogel may comprise NaCl. In other embodiments, the hydrogel may not comprise NaCl. The pore size of the hydrogel may be between 2 and 15 μm.

Also described herein are methods of treating a disease. In some variations, a method of treating a neurological disease may comprise implanting any of the hydrogels described herein into a subject. For example, methods may comprise implanting a hydrogel into the brain of a subject. In some embodiments, methods may comprise implanting the hydrogel into the striatum region of the brain of a subject, for example, the caudate putamen. In some embodiments, the hydrogel may be implanted into a subject suffering from Parkinson's disease. A subject may show an improvement in dopamine induced locomotor movements after hydrogel implantation. In some embodiments, modified stem cells contained in a hydrogel implanted into a subject according to methods described herein may be viable for at least approximately 15, 20, 25, 30, 35, 40, or greater than 40 days after implantation. The modified stem cells of the hydrogel may exhibit differentiation into functional neurons after implantation into a subject. The implanted modified stem cells may decrease, ameliorate, or prevent at least one symptom of a neurological disorder of a subject.

Also described herein are methods of preparing any of the hydrogels described above for implantation into a subject. A method of preparing a hydrogel for implantation into a subject may comprise culturing stem cells in media, providing a hydrogel generated using self-aggregating peptides and comprising one or more growth factors, loading the stem cells into the hydrogel, and implanting the hydrogel loaded with stem cells into the subject. In some variations, the stem cells may be cultured in media comprising a plurality of growth factors. In some variations, the self-aggregating peptides used to generate the hydrogel may be derived from an amyloid protein. Methods of preparing a hydrogel may comprise forming a hydrogel by mixing a powder form of self-aggregating peptides with a buffer solution. In some embodiments, methods may comprise sterilizing the buffer solution and/or sterilizing the peptide power. Sterilizing the buffer solution may comprise filtering the solution through a sterile syringe filter. Media used to culture stem cells may comprise SHH and FGF-8 growth factors. In some embodiments, the media may not contain the growth factor FGF-2. Stem cells may be mesenchymal stem cells. Hydrogels generated according to methods described herein may comprise cells at a concentration within the range of approximately 200-4000 cells per μg of peptide. The hydrogel may have a pH in the range of 6.5-7.5. In some embodiments, the hydrogel may comprise NaCl. In other embodiments, the hydrogel may not comprise NaCl. The pore size of the hydrogel may be between 2 and 15 μm. A subset of the stem cells contained in hydrogels generated according to methods described herein may express nestin and/or beta-III tubulin. In some embodiments, a subset of the stem cells may exhibit upregulated tyrosine hydroxylase expression. In some embodiments, modified stem cells contained in a hydrogel generated according to methods described herein may be viable for at least approximately 15, 20, 25, 30, 35, 40, or greater than 40 days after implantation. The modified stem cells of the hydrogel may exhibit differentiation into functional neurons after implantation into a subject. The implanted modified stem cells may decrease, ameliorate, or prevent at least one symptom of a neurological disorder of a subject.

### BRIEF DESCRIPTION OF THE DRAWINGS

FIG. 1 is a schematic depiction of a cross-β sheet structure.

FIG. 2 is an image of a hydrogel loaded with stem cells stained for nestin.

FIG. 3A shows tyrosine hydroxylase (TH) gene expression by qRT-PCR in differentiated stem cells as compared to housekeeping genes.

FIG. 3B shows a hydrogel containing stem cells stained for tyrosine hydroxylase.

FIG. 4A shows a coverslip containing stem cells in a control condition.

FIG. 4B shows a coverslip containing a hydrogel loaded with stem cells.

FIG. 5 shows normalized average velocity measurements at various time points pre and post hydrogel implantation by MPTP mice implanted with hydrogel composition “prep A.”

FIG. 6 shows normalized average velocity measurements at various time points pre and post hydrogel implantation by MPTP mice implanted with hydrogel composition “prep B.”

FIG. 7 shows normalized average vertical rearing observations at various time points pre and post hydrogel implantation by MPTP implanted with hydrogel composition “_{prep A.”}

FIG. 8 shows normalized average vertical rearing observations at various time points pre and post hydrogel implantation by MPTP implanted with hydrogel composition “prep B.”

FIG. 9A is a series of images of a brain section taken from the brain of a MPTP mouse treated with a two different hydrogel compositions at 7 days post hydrogel implantation, comprising fluorescent stained stem cells.

FIG. 9B is a series of images of a brain section taken from the brain of a MPTP mouse treated with a two different hydrogel compositions at 14 days post hydrogel implantation comprising fluorescent stained stem cells.

FIG. 9C is a series of images of a brain section taken from the brain of a MPTP mouse treated with a two different hydrogel compositions at 21 days post hydrogel implantation comprising fluorescent stained stem cells.

FIG. 10 shows the viability of SHSY5Y cells cultured for 24 hours in laminin derived hydrogels.

### DETAILED DESCRIPTION

Described herein are hydrogels, methods of preparing hydrogels, and methods of implanting hydrogels. The hydrogels described herein may be implanted into a subject and used to treat diseases. Optionally, in some embodiments hydrogels may comprise stem cells. Hydrogels implanted into a subject may be used to treat neurological diseases such as Parkinson's disease. Hydrogels described herein are comprised of self-aggregating peptides. Hydrogels and methods of forming hydrogels may comprise loading growth factors and/or stem cells into a hydrogel. Also described herein are methods of modifying stem cells for loading into a hydrogel. Methods of modifying stem cells may comprise culturing stem cells in media to achieve a desired state of differentiation, for example, towards a dopamine-producing neuron. Hydrogels comprising stem cells and/or growth factors may be implanted into a subject. For example, hydrogels comprising stem cells and/or growth factors may be implanted into the brain of a subject to treat a neurological disorder such as Parkinson's disease. Hydrogels described herein may contain stem cells, sustain stem cell viability, and encourage stem cells to grow and differentiate into neurons by providing mechanical and biochemical cues, thereby replacing dead, damaged, or dysfunctional neurons. Implantation of the hydrogels provided herein may provide the benefit of decreasing, ameliorating, or preventing at least one symptom of a neurological disorder.

### I. Hydrogels

Described herein are hydrogels comprised of self-aggregating peptides configured for implantation into a tissue or organ of a subject. For example, hydrogels described herein may be implanted into the central nervous system of a subject, such as into the brain or spinal cord. The hydrogels may be prepared to contain growth factors and/or stem cells. Self-aggregating peptides used to generate a hydrogel may be derived from proteins with a propensity to form cross-β-sheet rich structures. In some embodiments, self-aggregating peptides may be derived from amyloid proteins. In other embodiments, peptides may be derived from non-amyloid proteins. Also described herein are methods of preparing a hydrogel. In some embodiments, methods of preparing a hydrogel comprise loading growth factors into a hydrogel.

### A. Hydrogel Properties

Hydrogels described herein are comprised of self-aggregating peptides. Generally, without being bound to any theory or mechanism, hydrogels are a three-dimensional (3D) network made of polymer chains crosslinked physically or chemically that can entrap water or biological liquids. Peptide hydrogels are a class of hydrogels wherein the component peptides self-assemble to form an ordered nanostructure. These ordered nanostructures may then gather to form a supramolecular network, which can entrap water or other liquid for gelation. In some embodiments, the nanostructures may mimic characteristics of the extra-cellular matrix. The use of self-aggregating peptides in hydrogel formation may provide the benefit of resulting in gelation without the addition of chemicals that could be toxic when implanted into a subject. As generally used herein, self-aggregation or self-assembly refers to the ability of the peptides to assemble and form a stable structure.

Peptides used to form hydrogels described herein may have a propensity to form β-sheet structures, such as cross-β-sheet structures. In some embodiments, the peptides used to form hydrogels described herein may be derived from amyloid proteins. Amyloid proteins are a class of proteins with a propensity for forming highly repetitive cross-β sheet structures. In principle, amyloid structures may be adopted by many types of proteins under certain conditions. In this context, “derived from” may encompass both a peptide sequence extracted from a protein and a peptide sequence extracted from a protein and subsequently modified. Modification may encompass, for example, replacing one or more amino acids in the peptide sequence, and/or modifying the terminal end(s) of the sequence. In some embodiments, the peptides may comprise non-naturally occurring amino acids or synthetic amino acids. In some embodiments, the peptides may further comprise conjugated labels, tags, small molecules, polymers, and/or biomolecules at any position on the peptide. Using peptides derived from amyloid proteins may impart desirable aspects of amyloid proteins into the hydrogel. For example, the propensity to form highly repetitive and stable structures (e.g. cross β-sheet structures, as described above). Further, the hydrophobicity and charged residues in some sequences may make the surface of amyloid fibrils uniquely able to bind to both large macromolecules/polymers and small molecules and cells. In other embodiments, peptides used to form hydrogels may be derived from non-amyloid proteins. That is, proteins where the naturally occurring form of the protein may not form cross-β-sheet rich structures under physiological conditions. However, peptides derived from non-amyloid proteins may still have a propensity for form cross-β-sheet structures. For example, peptides used to form hydrogels described herein may be derived from an amyloidogenic stretch of a non-amyloid protein. That is, a peptide may be derived from a region of a non-amyloid protein that has a propensity to form cross-β-sheet structures.

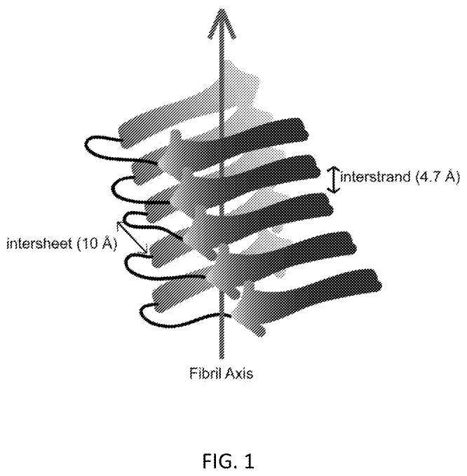

In some embodiments, the self-aggregating peptides used to form a hydrogel self-assemble into nano-fibers, and the nano-fibers form into cross-β-sheet structures when they reach a sufficient concentration. An exemplary embodiment of a cross-β-sheet structure is depicted in FIG. 1. As depicted in FIG. 1, the individual β-sheet strands are orthogonal, and the β-sheets are parallel to the fibril axis. This organization enables dense peptide packing, and may provide the benefit of forming highly ordered and stable hydrogels. Nano-fibers with higher-order structures like β-sheets may possess unique stability against various harsh environmental conditions such as wide ranges of pH, temperature, and proteases, making them well-suited for implantation into a subject. Higher-order alignment may also provide the benefit of imparting unique mechanical strength onto the hydrogels, which may aid in directing stem cell differentiation to neuronal lineage in embodiments wherein cells are contained in the hydrogels.

As described above, peptides used to form hydrogels described herein have a propensity to form cross-β-sheet structures. As contemplated herein, the peptides used to form hydrogels may be derived from proteins of Groups I, II, and III. As used herein, Group I refers to any of the following proteins: A-beta, alpha-synuclein, beta amyloid, Tau, Lysozyme, ApoA1, transthyretin, serum amyloid A, immunoglobulin light chain, beta-2 microglobulin, prion, and/or amylin. In some embodiments, peptides may be derived from a Group I protein(s), wherein the protein(s) is not alpha synuclein nor beta amyloid. Group II refers to any of the following proteins: Ure2, Sup35, Curlin, Het-S, secretory hormones (such as those listed in Table 1, below), SodC, Prio, Rxfp2 and/or pMel17. Group III refers to non-amyloid proteins (where the protein in its full length does not have a propensity to form cross-β-sheet structures, i.e. is not “amyloidogenic”). Some examples of Group III proteins are laminin, TARDBP, AQP2, MYO15, and AMPN. In some embodiments, a particular protein may be classified by more than one group. Exemplary peptides of this disclosure derived from amyloid proteins are shown in Table 2. Exemplary peptides of this disclosure derived from non-amyloid proteins are shown in Table 3.

TABLE 1

| | | | | |
|---|---|---|---|---|
| r/hCRF | sfUro | GIP | GRP | hPRL |
| oCRF | hGhrelin | Exendin4 | hACTH | α-CRF |
| hUcn | hGRF-40 | Helodermin | rACTH | |
| rUcn | hGRF44 | pGalanin | pACTh | |
| hUcnII | VIP | hGalanin | hβ-endorphin | |
| mUcnII | sCalcitonin | cPACAP | oβ-endorphin | |
| rucnII | Glucagon | Neuro K | Salmon MSH | |
| huCNIII | GLP-1 | Obestatin | Oxytonin | |
| mUcnIII | GLP-2 | Bombesin | AVP | |
| Sauv | Secretin | pNPY | Somatostatin | |

The peptides for generating hydrogels may be of any suitable length. For example, an amino acid sequence used to form a self-aggregating peptide for use in a hydrogel may be between 2- and about 21 amino acids. For example, a self-aggregating peptide for use in a hydrogel herein may be 2, 3, 4, 5, 6, 7, 8, 9, 10, 11, 12, 13, 14, 15, 16, 17, 18, 19, 20, or 21 amino acids long. In some embodiments, the peptide sequence used to form a hydrogel may be between 5-9 amino acids, between 5-12 amino acids, or between 5-15 amino acids. The amino acid sequence of the peptides used to form hydrogels may be any suitable sequence.

In some embodiments, the peptides used to form a hydrogel may be identical. However, in other embodiments, hydrogels may be formed using a heterogeneous mixture of different (i.e. non-identical) peptides. The peptides used to form hydrogels may comprise an amino acid sequence with a modified N-terminus and/or a modified C-terminus. For example, the peptides may comprise a fluorenylmethoxycarbonyl (F-moc or Fmoc) protected N-terminus. The F-moc protected N-terminus may provide the benefit of enhancing intermolecular π-π interactions of F-moc groups, which may facilitate peptides to come closer together and result in successful self-assembly and amyloid fibril formation.

In some embodiments, peptides used to form a self-aggregating hydrogel may be derived from an amyloid protein, by way of example, the amino acid sequence may be any of the sequences listed in Table 2.

In some embodiments, peptides used to form a self-aggregating hydrogel may be derived from the amyloid protein alpha-synuclein. Alpha-synuclein (“α-Syn”) is a 140 amino-acid protein that contains numerous regions of amyloidogenicity and exhibits a high propensity for cross-β-sheet formation. The peptides used to form a hydrogel may be derived from the NAC region of an α-Syn protein. Peptides derived from the NAC region of an α-Syn protein may provide the benefit of being highly prone to β-aggregation. In some embodiments, the peptide used to form a hydrogel may be comprised of a 5 amino acid sequence derived from the non-Aβ component (“NAC”) region of an α-Syn protein. For example, the amino acid sequence selected from within the NAC region of an α-Syn protein may be VTAVA (SEQ ID NO: 1). In some embodiments, the amino acid sequence VTAVA (SEQ ID NO: 1), selected from the NAC region of the α-Syn protein, may be modified by replacing one or more amino acids. For example, the peptide sequence may be modified to enhance the gelation ability of the hydrogel and/or to enhance the stacking properties of the peptides. The amino acid sequence of a peptide derived from a modified NAC region may not comprise a tyrosine amino acid. For example, in some embodiments, the amino acid sequence used in the formation of a hydrogel may be VHAVA (SEQ ID NO: 8).

Table 2 provides exemplary peptides.

TABLE 2

| SEQ ID NO: | SEQUENCE |
|---|---|
| 1 | VTAVA |
| 2 | VTVVA |
| 3 | VTAVV |
| 4 | VLAVA |
| 5 | VYAVA |
| 6 | VRAVA |
| 7 | VHAVV |
| 8 | VHAVA |
| 9 | VFAVA |
| 10 | VWAVA |

In some embodiments, the peptides of Table 2 further comprise an Fmoc protected N-terminus. In some embodiments, SEQ ID NO:1 comprises an Fmoc protected N-terminus. In some embodiments, SEQ ID NO: 2 comprises an Fmoc protected N-terminus. In some embodiments, SEQ ID NO: 3 comprises an Fmoc protected N-terminus. In some embodiments, SEQ ID NO: 4 comprises an Fmoc protected N-terminus. In some embodiments, SEQ ID NO: 5 comprises an Fmoc protected N-terminus. In some embodiments, SEQ ID NO: 6 comprises an Fmoc protected N-terminus. In some embodiments, SEQ ID NO: 7 comprises an Fmoc protected N-terminus. In some embodiments, SEQ ID NO: 8 comprises an Fmoc protected N-terminus. In some embodiments, SEQ ID NO: 9 comprises an Fmoc protected N-terminus. In some embodiments, SEQ ID NO: 10 comprises an Fmoc protected N-terminus.

Although peptides derived from an α-Syn protein were described in detail above, in some embodiments, peptides may be derived from any suitable protein. For example, peptides may be derived from other amyloid proteins, such as beta amyloid, alpha-beta, or any other suitable amyloid protein.

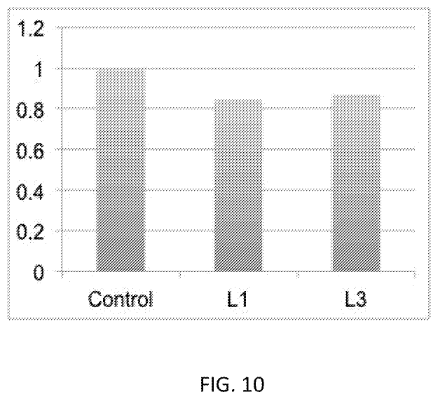

In other embodiments, peptides may be derived from non-amyloid proteins, by way of example, the peptide may comprise the any of the sequences listed in Table 3. For example, peptides may be derived from laminin. Laminin is a structural glycoprotein found in the basement membranes of tissues and exists in different forms in various tissues through diverse combinations of its three sub-chains. Peptides derived from laminin may be derived from portions of laminin that have a high propensity to form cross-β-sheet structures. That is, the peptides may be derived from an amyloidogenic stretch of a laminin protein. An amyloidogenic stretch of a protein may be identified by any suitable method, such as computational modelling, for example. Exemplary engineered peptide sequences, L1-L3, are shown in Table 3 below. Hydrogels formed by L1 and L3 displayed high viability of SHSY5Y cells relative to control when incubated for 24 hours, as shown in FIG. 10.

Table 3 provides exemplary peptides derived from a non-amyloid protein, laminin.

TABLE 3
Engineered peptides derived from
laminin for hydrogel formation.

| SEQ ID NO: | Sequence |
|---|---|
| 11 | LLFYL |
| 12 | LLLYL |
| 13 | LLVYL |

In some embodiments, the peptides of Table 3 further comprise an Fmoc protected N-terminus. In some embodiments, SEQ ID NO: 11 comprises an Fmoc protected N-terminus, referred to herein as L1. In some embodiments, SEQ ID NO: 12 comprises an Fmoc protected N-terminus, referred to herein as L2. In some embodiments, SEQ ID NO: 13 comprises an Fmoc protected N-terminus, referred to herein as L3.

In some embodiments, the engineered laminin peptides may have an amino acid sequence LLXYL (SEQ ID NO: 14), where X represents any amino acid. In some embodiments X=F, in some embodiments, X=V. In some embodiments SEQ ID NO: 14 may further have the N-terminus protected with an Fmoc moiety.

Hydrogels described herein may be configured to provide differentiation cues to encourage the stem cells contained within a hydrogel to differentiate toward a particular cell lineage. For example, hydrogels described herein may provide stems cells with both physical and biochemical signals that encourage differentiation towards neuronal lineage. In some embodiments, hydrogels may comprise growth factors. Growth factors may provide the benefit of inducing targeted differentiation in stem cells towards a particular lineage (e.g. neuronal lineage) by activating particular pathways via cell surface receptors. A hydrogel comprising growth factors may allow for sustained exposure of growth factors to the cells contained in the hydrogel. Sustained exposure may provide the benefit of facilitating the differentiation of stem cells into dopamine producing neurons, for example. Any suitable growth factor or combination of growth factors may be contained in a hydrogel. One or more of Sonic Hedgehog (SHH), Fibroblast Growth Factor 2 (FGF-2), and Fibroblast Growth Factor 8 (FGF-8) may be contained in the hydrogel. For example, in one embodiment, the hydrogel may contain the N-termini of SHH growth factors, and FGF-8. The pore size of the hydrogel may affect the mobility of the growth factors in the hydrogel, which may in turn impact the rate of diffusion of the growth factors. For example, a smaller pore size may result in slower migration of growth factors. In some embodiments, this may result in a longer period of stem cell exposure to growth factors within the hydrogel, which may affect differentiation of the stem cells. In some embodiments, the pore size of the hydrogel may be between 2 μm and 15 μm.

As described above, hydrogels may also provide mechanical cues to encourage stem cell differentiation toward a target cell lineage. Hydrogels may provide scaffolding and support for cells to anchor to as they differentiate. Additionally, because stem cells are mechanosensitive, the stiffness of their environment may influence the differentiation of stem cells. For example, soft hydrogels that mimic the softness of the extracellular matrix in the brain may promote stem cell differentiation towards neurons. Thus, the structure and stiffness of hydrogel described herein may be manipulated to provide mechanical cues that encourage stem cells to differentiate into neuronal cell. Hydrogels described herein may comprise a structure that mimics the softness of the extracellular matrix in order to encourage stem cells to differentiate toward neuronal cell lineages. Hydrogels described herein may also have properties that may be easily manipulated to mimic various environments. For example, hydrogels described herein may have a stiffness that is easily modulated such that hydrogels may be capable of mimicking extracellular matrices is various areas of the body.

Hydrogels described herein may comprise various properties which make the hydrogels desirable for implantation into a tissue or organ such as in a subject, and/or able to effectively contain and support growth factors and/or stem cells. For example, in some embodiments, hydrogels described herein may be thixotropic, or self-healing. That is, the hydrogel may exhibit reversible breaking when placed under mechanical stress, and re-form under static conditions. Hydrophobic surfaces of the proteins used to form the hydrogels described herein may contribute to the self-healing nature of the hydrogels. Self-healing may provide the benefit of allowing the gel to be implanted into a subject via minimally invasive means. For example, a needle or syringe may be used to implant the hydrogel into the brain or spinal cord of a subject, and the self-healing nature may allow the hydrogel quickly re-form into a structured gel after implantation. This may permit less invasive methods of implantation into a subject, for example, into the brain. Hydrogels described herein, particularly those comprising a cross-β sheet rich structure, may assemble into a nano-fiber meshwork mimicking the natural extracellular matrix. This may provide the benefit of encouraging the proliferation and differentiation of stem cells contained within the hydrogel.

Hydrogels described herein may also provide the benefit of inducing a minimal inflammatory response when implanted into a subject. That is, the inflammatory response induced by hydrogels described herein when implanted into a subject may subside in an acceptable amount of time. For example, when implanted into the brain of a subject, an inflammatory response evoked by hydrogels described herein may subside in a desirable time fame such as from 5-10, 10-15, 15-20, 20-25, 25-30, 30-35 days, or any acceptable range.

The hydrogels described herein may be biodegradable. Hydrogels may be configured to be stable (i.e. not degrade) for a timeframe long enough to promote stem cell survival and differentiation. Hydrogels may degrade within any suitable timeframe, for example between 20 and 30 days post implantation. In other embodiments, hydrogels may degrade between 50 and 70 days post implantation.

The hydrogel may also provide the benefit of retaining stem cells to a particular location within a tissue or organ of a subject. A hydrogel comprising stem cells may promote localized differentiation of the stem cells, and lessen cell migration to undesirable areas of the subject, such as to different tissues, organs, or other locations within tissues or organs that are not intended to be treated. The hydrogel may also sustain the viability of stem cells. As used herein, “viability” refers to the ability of stem cells to remain metabolically active for growth and neural function. In some embodiments, hydrogels may sustain the viability of stem cells contained therein after implantation of a hydrogel into a subject (e.g. into the brain). Stem cells loaded into a hydrogel and implanted into a subject may remain viable for any suitable period of time. For example, hydrogels may sustain the viability of stem cells contained therein for at least 7, 15, 20, 25, 30, 35, 40, or greater than 40 days after implantation into a subject. In some embodiments, hydrogels may sustain the viability of stem cells contained therein prior to implantation, such that the hydrogels containing the stem cells can be stored and/or cultured in vitro, for example prior to implantation. For example, hydrogels may sustain the viability of stem cells contained therein for at least 5, 10, 15, 20, or greater than 20 days in vitro.

Hydrogels described herein may also have properties that may be precisely controlled to achieve a desired functionality. For example, the pore size and stiffness of the hydrogel may be manipulated by the addition of a salt. In some variations, salts (e.g. NaCl) may be added to a hydrogel to decrease the pore size of the hydrogel. This may provide the benefit of controlling the rate of diffusion of growth factors throughout the hydrogel, for example.

The hydrogels for implantation into a subject described herein may exhibit any combination of the properties described above.

### B. Methods of Making a Hydrogel

Also described herein are methods of making a hydrogel. Methods described herein may comprise mixing a peptide stock with a buffer solution, and allowing the mixture to self-gel. In some embodiments, the peptide stock may be peptide in powdered form. In other embodiments, the peptide may be maintained as a stock solution, and aliquots of stock solution may be mixed with a buffer solution in order to form a hydrogel. Any suitable peptide, such as those described above, may be used to form a peptide stock used in the formation of a hydrogel. In some embodiments, peptides derived from amyloid proteins such as α-Syn may be used to form a peptide stock. For example, a peptide stock may comprise any one of the peptide sequences derived from an α-Syn protein in Table 2, as described above. In other embodiments, a peptide sequence derived from a non-amyloid protein, such as laminin, may form the peptide stock used to form a hydrogel. For example, a peptide stock may comprise any one of the peptide sequences derived from laminin in Table 3. As described above, the peptide stock used to form a hydrogel may be comprised of a plurality of identical peptides, or it may be comprised of a mixture of different peptides. Methods of making a hydrogel may comprise modifying an N-terminus or a C-terminus of the peptide. For example, methods of making a hydrogel may comprise modifying a peptide to include an F-moc protected N-terminus.

Any suitable buffer solution may be used in the formation of a hydrogel. For example, methods of forming a hydrogel may comprise mixing a peptide stock with a phosphate buffer such as 20 mM phosphate buffer. Any suitable phosphate buffer may be used, for example, buffers in the range of 2 mM-20 nM. In other embodiments, methods of forming a hydrogel may not use a phosphate buffer, but may instead comprise mixing a peptide stock with water. In some embodiments, the method of making a hydrogel may comprise dissolving the peptide stock in the buffer by heating the buffer-peptide mixture. In other embodiments, dissolving the peptide stock in the buffer solution may comprise altering the pH of the solution, for example, by adding a base such as NaOH. However, any suitable acid or base may be used to alter the pH of the solution. In some embodiments, dissolving the peptide in the buffer may not comprise adding additional elements, and may simply comprise mixing the peptide stock with the buffer. Any suitable method of mixing a buffer solution with a peptide stock may be used, such as vortexing or stirring.

Methods of forming a hydrogel may comprise employing one or more processes to encourage gelation. In some embodiments, methods of forming a hydrogel may comprise temperature changes, such as utilizing a heating and cooling cycle to encourage gelation. Any suitable number of heating and/or cooling cycles may be used. For example, three heating and cooling cycles of the peptide-buffer mixture may be used to form a gel. In some embodiments, changes in pH may be used rather than, or in addition to, temperature changes to facilitate gelation of the peptide-buffer mixture. For example, methods of forming a hydrogel may comprise raising the pH of the buffer-peptide solution, and subsequently lowering the pH to encourage gelation. Any suitable number of pH raising and lowering cycles may be employed in forming a hydrogel. In some embodiments, methods of forming a hydrogel may comprise adding salt to the peptide-buffer mixture. In some embodiments, gelation may not occur without addition of salt. However, in other embodiments, gelation may be achieved without salt. Even where gelation may be achieved without salt, methods of forming a hydrogel may comprise adding salt. For example, methods of forming a hydrogel may comprise adding NaCl in order to affect the pore size of the gel. Addition of salt may decrease pore size. Thus, methods of making a hydrogel may comprise adding NaCl until a desired pore size is achieved. For example, methods may comprise adding NaCl to the hydrogel such that the hydrogel comprises a concentration of NaCl in the range of 50 mM to 150 mM. Any suitable salt may be used to affect pore size of a hydrogel. For example, CaCl₂ in the range of 25 mM to 75 mM may be used to affect the pore size of a hydrogel. In some embodiments, the pore size of the hydrogel may be less than about 20 μm. In some embodiments, the pore size of the hydrogel may be between about 5 μm and about 15 μm or between about 5 μm to about 10 μm. As described herein, pore size may refer to an average diameter of the pores of a hydrogel. A desirable pore size may result in an effective level of diffusion of growth factors through the hydrogel, and a desired rate of sustained diffusion of growth factors (or other proteins) as the hydrogel is degraded. In some embodiments, the addition of salt may be used to affect the stiffness of the hydrogel. For example, methods may comprise adding salt until a desired level of rigidity or stiffness is achieved.

Methods of making a hydrogel may further comprise altering the pH of the hydrogel to a desired level. For example, methods of making a hydrogel may comprise adding HCL. However, any suitable acid or base may be used to alter the pH of a hydrogel. Methods may comprise reaching any suitable pH, for example, a pH in the range of 6.5-7.5. This range may be desirable because it may approximate the pH in vivo at the implantation site. Although methods described above involve mixing a peptide stock with a buffer solution, and allowing self-gelation, any suitable method of forming a gel may be used. For example, a portion of the peptide power may first be mixed with a buffer solution and allowed to gel, and the remainder of the peptide power may be added after gelation is complete. In other embodiments, the peptide may be in the form of a liquid rather than a powder (as described above), and forming a hydrogel may comprise mixing the liquid peptide solution with the liquid buffer. Further, chemical gelation aids may be used in the formation of the gel.

Methods of forming a hydrogel may also comprise sterilizing various components of the gel mixture. Sterilization processes may provide the benefit of allowing for safe and sterile implantation into a subject. Sterilization of hydrogel components may occur at various points in the formation of a hydrogel. In some embodiments, the buffer may be sterilized prior to mixing with other gel components. For example, the buffer used in the formation of the hydrogel may be sterilized using a sterilized strainer or filter such as a syringe filter. In some embodiments, the peptide mixture may be sterilized instead of or in addition to sterilization of the buffer solution. For example, the peptide stock used to form the hydrogel may be sterilized prior to mixing with the buffer. The buffer/peptide stock mixture may also be sterilized prior to or after gelation. In other embodiments, other components of the hydrogel may be sterilized separately or together. For example, growth factors, differentiation media, or a hydrogel containing some of all of these components may be sterilized. The components of the hydrogel may be sterilized by any suitable method, including temperature changes, and/or UV light mediated sterilization (e.g. prior to the addition of cells for culture).

Methods of forming a hydrogel may also comprise loading growth factors into the hydrogel. Any suitable growth factor or combination of growth factors may be loaded into a hydrogel. In some embodiments, a method of forming a hydrogel may comprise loading one or more of the growth factors, such as FGF-8, SHH, FGF-2, B27, GDNF and/or BDNF into a hydrogel. The SHH growth factor may comprise a portion of the growth factor, for example, only the N-terminus. In some variations, the hydrogel may comprise one of FGF-8, SHH, FGF-2, B27, GDNF or BDNF. In other variations the hydrogel may comprise a combination of growth factors, such as FGF-8 and SHH (N-terminus), FGF-8 and FGF-2, FGF-8 and B27, FGF-8 and BDNF, SHH and FGF-2, SHH and B27, SHH and BDNF, FGF-2 and BDNF, FGF-2 and B27, or B27 and BDNF. In other variations, the hydrogel may comprise a combination of two or more growth factors, such as FGF-8, SHH and B27; FGF-8, SHH, and FGF-2; FGF-8, SHH, and BDNF, for example.

Growth factors may be loaded into the hydrogel in any suitable manner. In one embodiment, a hydrogel is first provided according to methods described above, and the hydrogel is then vortexed and mixed with growth factors. The hydrogel may temporarily be liquefied via vortexing, and reform into a hydrogel after the growth factors are mixed with the hydrogel. Gelation after addition of growth factors may be facilitated, for example, by casting the hydrogel and growth factor mixture on a coverslip and leaving the mixture undisturbed. However, the self-healing properties of the hydrogel may be sufficient for the gel to reform after vortexing with growth factors regardless of any additional steps. Methods of loading growth factors into a hydrogel may result in a substantially even distribution of growth factors within a hydrogel In other embodiments, methods of loading growth factors within a hydrogel may result in an uneven, or localized distribution of growth factors within a hydrogel. For example, growth factors may be loaded into the gel by a needle or syringe to deliver growth factors to only a portion of the gel. In other embodiments, growth factors may be mixed into the hydrogel prior to gelation (i.e. mixing into a solution that has yet to form a gel). Mixing growth factors into the hydrogel prior to gelation may result in even or uneven distribution of growth factors. Further, in some embodiments, a gradient of growth factors may be created in the gel by exposing the hydrogel and/or stem cells implanted into the hydrogel to differentiation media. For example, growth factors can be contained in a media in which a hydrogel stem cell construct is cultured. That is, a hydrogel (for example, a hydrogel comprising stem cells) may be cultured in a dish with differentiation media comprising growth factors. Growth factors may diffuse from media into the gel without the step of mixing the growth factors into the gel. Loading growth factors into a hydrogel may provide the benefit of aiding the hydrogel in maintaining the viability of stem cells and/or encouraging stem cells to differentiate towards a target cell type.

### II. Stem Cells

Described herein are compositions comprising hydrogels loaded with stem cells prior to implantation in a subject. In some embodiments, stem cells may be modified prior to loading into the hydrogel. For example, it may be desirable for stem cells to be partially differentiated prior to loading into the hydrogel. Thus, methods of modifying stem cells may comprise culturing stem cells in media comprising with growth factors to encourage stem cell differentiation. When implanted into a tissue or organ, the hydrogel comprising the stem cells may further encourage growth and differentiation of the stem cells, for example, into differentiated cells. For example, when implanted into the brain or spinal cord, the hydrogel comprising the stem cells may further encourage growth and differentiation of the stem cells, for example, into neuronal cells.

### A. Stem Cell Properties

Any suitable type of stem cell may be utilized for loading into a hydrogel, and subsequently implanted into a tissue or organ of a subject. In some embodiments, stem cells implanted into a hydrogel may be mesenchymal stem cells, for example, human mesenchymal stem cells. In one embodiment, bone marrow derived mesenchymal stem cells (BM-hMSCs) may be used for implantation into a hydrogel. BM-hMSCs may beneficial because of their ability to promote tissue repair via various mechanisms, such as secreting soluble factors and stimulating proliferation and differentiation of stem cells. Further, BM-hMSCs may provide the benefit of decreased inflammatory reactions when implanted into a subject. However, various types of stems cells may be used for loading into a hydrogel. For example, embryonic, pluripotent, induced pluripotent, or neural stem cells may also be used for loading into a hydrogel.

### B. Modification of Stem Cells

Stem cells may be modified prior to loading into a hydrogel, e.g. to generate partially differentiated stem cells, e.g. stem cells committed to generating neurons (“neural stem cells”). Accordingly, in some embodiments, stem cell modification may comprise priming stem cells to encourage differentiation of the stem cells toward a specific cell lineage. In some variations, methods of priming stem cells may encourage differentiation of stem cells to a neuronal lineage, for example. Priming stem cells may comprise culturing the stem cells in a media comprising one or more growth factors (“differentiation media”). In other embodiments, priming stem cells may comprise culturing stem cells in a hydrogel. For example, stem cells may be cultured in a hydrogel exposed to differentiation media. Exposing a hydrogel to differentiation media comprising growth factors may provide the benefit of creating a gradient of growth factors throughout the hydrogel. The hydrogel stem cell culture may be a 2D cell culture or a 3D cell culture. Stem cells may be cultured in a hydrogel with or without growth factors. In some embodiments, stem cells may be cultured in media and subsequently cultured in a hydrogel, or vice versa.

Different combinations of growth factors in base media may encourage differentiation towards various cell lineages. Culturing media may be comprised of a base media and one or more growth factors. In some embodiments, the base media may be a neurobasal media. However, the base media may be any suitable media, such as Dulbecco Modified Eagle Medium (“DMEM”), F12, alpha minimum essential media (“α-MEM”), or any combination thereof (e.g. a 50/50 mixture of DMEM and F12 media). In some embodiments, stem cells may be cultured in a media comprising one or more of the growth factors SHH, FGF-8, fibroblast growth factor-2 (FGF-2), and/or brain derived neurotropic factor (BDNF). In some embodiments, it may be desirable for differentiation media to not include FGF-2. For example, differentiation media used to culture stem cells may comprise the growth factors SHH (N-terminus) and FGF-8. Stem cells may be cultured in the presence of other cellular or non-cellular components in addition to growth factors, such as telomerase immortalized midbrain astrocytes. Differentiation media may also comprise supplements such as B27 and/or N2. Additional components may, for example, aid in encouraging stem cell viability, differentiation, and/or help stabilize terminal differentiating cells. Any suitable combination of growth factors may be used to culture stem cells and/or encourage differentiation toward a specific cell lineage. In some embodiments, the differentiation media may be configured to encourage stem cell differentiation toward neuronal lineage. For example, the differentiation media described above, comprising FGF-8 and SHH (N-terminus), has been shown to encourage stem cell differentiation towards neuronal lineage. Differentiation media may contain growth factors in any suitable concentration. For example, differentiation media may comprise approximately 0.0001 percent FGF-8 by volume, and 0.00025 percent SHH (N-terminus) by volume. In other variations, differentiation media may comprise between 0.0001 and 1 percent FGF-8 by volume, and/or between 0.00025 and 2.5 percent by volume.

Any suitable cell culturing method may be used to generate modified stem cells prior to implantation. Cells may be cultured and primed simultaneously, and the use of one term should not be understood to exclude the other. In some embodiments, methods of culturing stem cells may comprise seeding cells in expansion media and culturing cells for 24 hours prior to exposure to differentiation media. This may provide the benefit of reviving cells, if, for example they arrive in a frozen state. Cells cultures may be split and the cells seeded at any suitable density for culturing. For example, cells may be seeded at a density of approximately 50-150 cells per square centimeter. However, cells may be seeded at any suitable density range, for example, between 20-200, 20-50, 50-100, 100-150, 150-200, 200-250, 250-300, or any suitable density range. In some embodiments, cells are cultured until they reach 80% confluency. After reaching 80% confluency, cells may be further harvested and re-seeded for additional culturing. However, cells may be cultured and/or reseeded once they reach any suitable level of confluency, for example, 20-30%, 30-40%, 40-50%, 50-60%, 60-70%, 70-80%, 80-90% etc. Cells may also be cultured for any suitable amount of time. For example, cells may be cultured for 1-5, 5-10, 10-15, 15-20, 20-25, 25-30, 30-35 days, or for any suitable amount of time. Cells may be cultured in any suitable enclosure, such as a flask, petri disk, well plate, roller bottle, or larger bioreactor.

Cells may be cultured in differentiation media according to any suitable method. As described above, stem cells may be cultured directly in differentiation media, or stem cells may be cultured in a hydrogel, which may be exposed to differentiation media. In other embodiments, stem cells may be cultured in a hydrogel not exposed to differentiation media. The hydrogel may or may not comprise growth factors. Exposing a hydrogel to differentiation media (e.g. by storing the hydrogel in a well or plate comprising differentiation media) may create a gradient of growth factors within the hydrogel. In some embodiments, hydrogels may be cultured in differentiation media for a first period of time, and subsequently cultured in a hydrogel for a second period of time, or vice versa. In some embodiments, the differentiation media may be changed at one or more points during the cell culturing timeframe. Differentiation media may be changed in embodiments where cells are cultured directly in media, or where cells are cultured in a hydrogel exposed to media. For example, half of the media may be changed part-way through culturing, and replaced with fresh media of the same composition. In other embodiments, half of the media may be changed part-way through culturing, and replaced with a different media formulation. In other embodiments, the full amount of medial may be replaced part way through culturing, with either the same or a different media formulation. In other embodiments, the differentiation media may not be changed for the entire duration of cell culturing. Media may be changed at any suitable time point. For example, media may be changed after a particular number of days (e.g. half media may be changed once every 7 days). In other embodiments, media may be changed according to the cell density or percent confluency (e.g. half media changed once cells reach 50% confluency).

Methods of modifying stem cells described herein may comprise culturing stem cells in differentiation media until they reach a desired state of differentiation. It may be desirable to remove cells from differentiation media, and load them into a hydrogel for implantation into a subject, before they are fully differentiated (i.e. before they have fully formed into a particular cell type). In some embodiments, it may be desirable to implant stem cells into a subject in a partially differentiated state, such that the modified cells retain the ability for growth and differentiation once the cells are implanted into a target location in the subject. For example, in embodiments where stem cells are primed towards dopamine producing cells, it may be beneficial to culture cells in differentiation media until they exhibit certain dopamine producing markers. Such markers may signal that cells are in a partially differentiated state, but committed to developing into neuronal cells. Thus, methods of culturing stem cells for use in hydrogels described herein may comprise measuring various markers. In some embodiments, the ability of cells to continue growing and differentiating (e.g. into neuronal cells) may be retained for at least 20, 30, 40, 50, 60, 70, or 80 days after implantation. However, any suitable range may be suitable for cells to retain the ability to grow and differentiate.

Dopamine producing markers may include the expression of a particular protein or a particular cell morphology, for example. In some embodiments, methods may comprise culturing cells in differentiation media until they exhibit a bipolar elongated morphology, but not up to a point where cells exhibit branching. Branches on fully or partially differentiated stem cells may become damaged upon implantation into a subject via needle. Therefore, it may be beneficial for cells to remain in a non-branched state prior to implantation in order to avoid damage upon implantation. Cells may exhibit desirable markers of differentiation at any point in time, and some cells may progress towards differentiation faster than others. Thus, methods may comprise prescribing a desirable percentage or number of cells that exhibit a particular marker to determine when to remove cells from differentiation media and implant them into a hydrogel. For example, methods may comprise removing cells from differentiation media and implanting the cells into a hydrogel when greater than 50 percent of cells exhibit a particular marker. The threshold portion of cells exhibiting a marker of differentiation before the cells are removed from differentiation media may be any desirable percentage, such as 30 percent, 40 percent, 50 percent, 60 percent, 70 percent etc. Any suitable time frame may be utilized to culture cells until they reach a desirable state of differentiation. For example, it may be the case that it takes approximately 5 days for a predetermined percentage of stem cells to reach a desired state of differentiation. In such a case, stem cells may be removed from differentiation media after approximately 5 days, and implanted into a hydrogel for implantation into a subject. However, stem cells may differentiate to a desired state in any suitable time frame. For example, stem cells may be removed from differentiation media after 2-5 days, 5-10 days, 10-15 days, 15-20 days, 20-25, days, 25-30 days, 30-35 days, 35-40 days, 40-45 days etc. In other embodiments of methods for forming a hydrogel, stem cells may be removed from differentiation media without regard to their state of differentiation. For example, cells may be cultured for a prescribed number of days, or until a prescribed confluency.

Various indicators may be used to determine whether stem cells have differentiated to a desired state. Although cell morphology was used as an exemplary indicator above, other indicators may also signal the state of differentiation of stem cells. For example, protein expression may be used as an indicator of differentiation. In some embodiments, nestin expression may be used to determine when stem cells should be removed from differentiation media and/or implanted into a subject. Methods may comprise observing or measuring nestin expression in stem cells using any suitable method to determine when to remove stem cells from differentiation media. Nestin is an intermediate filament protein expressed in dividing cells during the early stages of development in the nervous system. Therefore, nestin may serve as an early indicator that a stem cell has committed to becoming a neural cell, but has not yet matured completely. In some embodiments, beta-III tubulin may be used as a marker to determine the state of differentiation of stem cells. Expression of beta-III tubulin correlates with early phases of neuronal phases of differentiation, and may also serve as an early indicator that a stem cells has committed to becoming a neuronal cell. In some embodiments of methods described herein, tyrosine hydroxylase (TH) may be used as a marker to determine the state of differentiation of stem cells. Methods may comprise observing or measuring TH expression in stem cells using any suitable method to determine when to remove stem cells from differentiation media. Any combination of proteins may be used to determine when cells are at a desirable state of differentiation. Cell markers indicative of stem cell differentiation towards a target cell lineage may be measured by any suitable method. For example, immunostaining techniques may be used to determine the presence of cells markers. However, it may also be the case that certain markers are predictably expressed by stem cells cultured in a particular way at a particular time point. Thus, in some embodiments, it may be possible to classify cell differentiation based on timing.

### C. Hydrogels Loaded with Stem Cells

Compositions described herein may comprise hydrogels loaded with stem cells. Stem cells modified according to methods described above may be loaded into a hydrogel in any suitable manner. Hydrogels may comprise stem cells at any suitable density or ratio, as described below. Hydrogels loaded with stem cells may or may not also comprise growth factors. Hydrogels loaded with stem cells and/or growth factors may be implanted into a tissue or organ of a subject, for example, into the central nervous system (e.g. the brain and/or spinal cord) of a subject, in order to treat a neurological disease.

### III. Methods of Use

Provided herein are methods of preparing any of the hydrogels of the disclosure for implantation, and methods of treatment.

### A. Preparing a Hydrogel for Implantation

Hydrogels configured for implantation into a subject may comprise stem cells, such as modified stem cells described herein. In other embodiments, hydrogels may be configured for implantation without stem cells. In some embodiments, methods of preparing a hydrogel for implantation may comprise loading stem cells, such as modified stem cells, into a hydrogel. Hydrogels may act to contain, sustain, provide differentiation cues to, and structurally support stem cells when implanted into a subject. For example, hydrogels may provide important mechanical cues to promote further stem cell differentiation, and growth factors contained within hydrogels may provide biochemical cues to promote stem cells differentiation. Methods described herein may comprise loading stem cells into a hydrogel, and implanting the hydrogel into a subject in order to treat a disease. Prior to loading into the hydrogel, stem cells may be modified according to methods described above (e.g. culturing cells in differentiation media to a desired level of differentiation). In some embodiments, hydrogels may also comprise growth factors. Growth factors may provide the benefit of maintaining the viability of stem cells as well as encouraging stem cells to differentiate into target cells (e.g. into dopamine producing neurons). In some embodiments, hydrogels may not comprise stem cells. For example, methods of treating a disease may comprise implanting a hydrogel into a subject, wherein the hydrogel does not comprise stem cells. The hydrogel alone may be beneficial to treatment of certain diseases by encouraging growth and/or healing of existing cells, for example. Hydrogels that do not contain stem cells may contain growth factors. A hydrogel that does not include stem cells may still provide benefits to existing stem cells at the implantation site, such as containing and sustaining stem cells, and providing mechanical and biochemical cues to encourage differentiation into a target cell type.

In some embodiments, stem cells may be loaded into the hydrogel at a density sufficient to result in effective treatment of disease when implanted into a subject, but low enough to avoid the toxicity associated with overabundant cell death. Cell death may result in the release of biochemicals, such as cytokines, growth factors, and/or hormones, etc. that may produce a toxic environment in the body in certain quantities. Therefore, it generally may be desirable to implant as few stem cells as are needed for therapeutic treatment. The use of hydrogels in treating neurological diseases may provide the benefit of allowing for effective therapeutic treatment with a decreased number of stem cells by improving stem cell viability, encouraging differentiation, and/or containing stem cells to a target area. Less cell death in combination with sustained localization of the stem cells may allow for a lower number of cells to be implanted to realize a therapeutic effect, without the risk of toxicity due to cell death. An effective density may vary for different cell types and different diseases.

In some embodiments, an effective density/ratio of cells may be 200-4000 stem cells per μg of peptide powder used to formulate a hydrogel. In some embodiments the density/ratio of cells is or is about 200, 250, 300, 350, 400, 450, 500, 750, 1000, 1250, 1500, 2000, 2500, 3000, 3500 or 4000 per μg of peptide powder used to formulate a hydrogel. As described above, hydrogels may provide the benefit of maintaining stem cell viability by releasing growth factors to sustain the stem cells and/or by providing an environment conducive to stem cell growth and differentiation. Hydrogels described herein may facilitate the maintenance of stem cell viability and differentiation when implanted into a subject for 15, 20, 25, 30, 35, 40, or greater than 40 days after implantation. In some embodiments described herein, the hydrogels facilitate maintenance and differentiation of the stem cells when implanted. The stem cells may differentiate and produce functional neurites. The stem may differentiate and become functional neurons.

Methods of preparing a hydrogel for implantation may comprise loading stem cells into a hydrogel in any suitable manner. In some embodiments, stem cells may be removed from differentiation media and formed into a pellet (e.g. by centrifuging), and vortexed with the hydrogel. In some embodiments, stem cells or a mixture comprising stem cells may be mixed into the hydrogel without formation of a pellet. In other embodiments, stem cells suspended in media or buffer may be loaded into a hydrogel via needle or syringe. Loading stem cells into the hydrogel via needle or syringe may provide the benefit of loading the stem cells in a specific location in the hydrogel. Methods may comprise implanting the hydrogel into a subject directly after cells are loaded into the hydrogel. In other embodiments, the hydrogel loaded with stem cells may be stored for some period of time prior to implantation. For example, the hydrogel may be kept overnight prior to implantation into a subject. Allowing stem cells to remain in the hydrogel for some period of time prior to implantation may provide the benefit of allowing the hydrogel to self heal from any damage causing during stem cell loading, and/or may allow stem cells to distribute throughout the hydrogel. In some embodiments, methods may comprise culturing stem cells in the hydrogel for some time prior to implantation into a subject. Allowing stem cells to differentiate and/or proliferate in the hydrogel may encourage further stem cell differentiation. Hydrogel stiffness and the fibers that make up the hydrogel may provide important mechanical signals that encourage stem cell differentiation. Therefore, culturing stem cells in the hydrogel, for some period of time, prior to implantation may encourage additional stem cell differentiation to a desired level. Although described above were methods of culturing cells to a desired level of differentiation in differentiation media prior to implantation to a hydrogel, some portion of culturing and/or priming may be done within the hydrogel.

### B. Methods of Treatment

Provided herein are methods of treating a neurological disease in subject in need thereof, comprising implanting into the subject any of the hydrogels described herein. The hydrogels may comprise growth factors and/or stem cells. In some embodiments, the hydrogel is implanted into the brain of the subject, in other embodiments, the hydrogel is implanted into the spinal cord of the subject.

Hydrogels described herein may be used to treat a neurological disease without the use of stem cells. In some embodiments, hydrogels without stem cells may comprise growth factors. In other embodiments, hydrogels may not comprise growth factors. Methods of using a hydrogel without stem cells to treat a neurological disease may comprise implanting the hydrogel into a subject. For example, hydrogels may be effective in treating neurodegenerative diseases without the use of stem cells by encouraging growth and proliferation of cells in a diseased region, or facilitating the repair of damaged cells. In some embodiments, hydrogels may provide a therapeutic effect by delivering growth factors to a diseased region to encourage cell growth, proliferation, differentiation, or repair. In other embodiments, the hydrogel alone may provide a therapeutic effect. For example, a hydrogel may provide mechanical cues to facilitate cell growth, proliferation, and differentiation and/or may constrain existing cells to a desired location to aid in increasing the number of cells at the implantation site.

Hydrogels described herein, when implanted into a subject, may be configured to treat a neurological disease. In some embodiments, hydrogels may be used to treat a neurological disorder. When implanted into the brain or spinal cord of a subject, a hydrogel may decrease, ameliorate, and prevent one or more symptoms of a neurological disorder. For example, hydrogels may be implanted into a brain of a subject for the purpose of treating a neurodegenerative disease such as Parkinson's disease. Hydrogels comprising stem cells may maintain the viability of the stem cells, and facilitate their growth and differentiation into dopamine-producing neurons in a diseased region of the subject. In some embodiments, hydrogel implantation may result in an increase in dopamine-producing neurons in the brain. This may result in an improvement in dopamine induced locomotor movements. The therapeutic effect of effects of a hydrogel implanted into a subject may result in behavioral recovery (i.e. decreased disease symptoms) between approximately 5-10, 10-15, 15-20, 20-25, 25-30, 30-35, 35-40, 40-45, or greater than 45 days after implantation. In some embodiments of compositions and methods described herein, hydrogels may be implanted into the spinal cord of a subject. In some neurological diseases, degradation, damage, or dysfunction of neurons may occur in the spinal cord, rather than the brain. Therefore, it may be beneficial to implant a hydrogel into the spinal cord of a subject to ameliorate one or more symptoms of a neurological disorder. In some embodiments, hydrogels described herein may be implanted into a subject alone. However, in other embodiments, hydrogels may be accompanied by an additional therapeutic when implanted into a subject, such as a drug.

Hydrogels may be implanted into any suitable subject. The hydrogel may be implanted into a human subject, for example. In other embodiments, the subject may be a non-human mammalian subject. For example, the subject may be a rodent (e.g. rat or mouse), primate, pig, companion animal (such as a dog or cat) or any suitable mammalian subject. Implanting a hydrogel into a non-human subject may be beneficial for testing hydrogel configurations and dosing.

The hydrogel comprising stem cells may be implanted into a subject in any suitable manner. In some embodiments, the hydrogel may be implanted into a tissue or organ of the subject, such as the brain or spinal cord. In some embodiments, the hydrogel may be implanted into a specific sub-location of a tissue or organ of a subject. For example, where a hydrogel is used to treat a neurodegenerative disease such as Parkinson's disease, the hydrogel may be implanted into the substantial nigra region of the brain of a subject. In some embodiments, the hydrogel may be implanted into the striatum region of the brain of a subject, for example, the caudate putamen. A needle, syringe, or cannula may be used to implant the hydrogel into the subject. For example, methods of implanting a hydrogel into brain tissue of a subject may comprise drilling an opening into the skull of a subject, and extending a needle/syringe/cannula through the opening in the skull, and into a desired region of the brain. In some embodiments, the needle may be temporarily left in the brain of the subject after the hydrogel is implanted to allow the construct to re-gel. For example, the needle may be left in the brain for a period of 5-15 minutes to permit the healing of the hydrogel. Shearing forces from the needle or syringe may cause the hydrogel to liquefy, and it may be beneficial to allow the construct to re-gel prior to removing the needle.

### IV. Kits

Also provided herein are kits for forming and/or using an implantable hydrogel. In some embodiments, kits may comprise one or more of the components used to form an implantable hydrogel, such as peptide powder/stock solution, buffer solution, differentiation media, stem cells, growth factors, salts, and/or any equipment that may be used in formation of a hydrogel. In other embodiments, kits may comprise a pre-formed hydrogel, which may or may not comprise stem cells and/or growth factors. In embodiments where a pre-formed gel is provided without growth factors and/or stem cells, either may be provided in the kit for loading into the hydrogel.

In embodiments of kits where a pre-formed gel is not provided, kits may comprise components for formation a hydrogel. For example, kits may comprise peptide powder/stock solution, buffer solution, and/or salt to form a hydrogel. Hydrogels formed by the components provided in kits described herein may be any of the hydrogel compositions described above, and may be formed according to any of the methods described above. In some embodiments, peptide stock provided by kits described herein may be comprised of peptides derived from amyloid proteins. For example, peptides may be derived from the amyloid protein α-Syn. In other embodiments, peptides may be derived from non-amyloid proteins. For example, the peptides may be derived from the non-amyloid protein laminin. Exemplary laminin derived peptides are shown in Table 3. The peptide stocks described herein may be comprised of any suitable peptide, such as, for example, the peptides listed in Table 2 and Table 3. Peptide stocks may be homogenous (i.e. contain peptides comprising only one sequence) or heterogeneous (i.e. contain peptides comprising two or more unique sequences). Any suitable buffer solution may be provided for mixing with the peptide stock, as described above. Kits may also comprise salts used in the formation of the hydrogel, as described above. For example, NaCl may be provided to affect the pore size of the hydrogel. Kits may further comprise any suitable equipment and/or machinery for formation of a hydrogel. For example, kits may comprise flasks, vials, beakers, dishes, well plates, coverslips, syringes, pipettes, scales, centrifuges, or any other suitable components. In some embodiments, kits may comprise equipment for sterilization of hydrogels such as a filter and/or syringe.

In embodiments of kits either wherein a pre-formed hydrogel is provided or wherein a pre-formed hydrogel is not provided, the kits of the disclosure may comprise growth factors, stem cells, and/or differentiation media. Growth factors may be provided for the purpose of loading into a hydrogel according to any of the methods described herein. Kits may comprise any suitable growth factors for loading into a hydrogel. For example, kits described herein may comprise FGF-8, SHH, and/or FGF-2. Kits may comprise any suitable type of stem cell, as described above. For example, kits may comprise mesenchymal stem cells. Stem cells provided by kits described herein may frozen for transportation, and therefore may require revival prior to implantation in a hydrogel. In some embodiments, kits may further comprise media for use in stem cell revival. Kits may further comprise differentiation media. Differentiation media may be used, for example, to modify stem cells by culturing the stem cells to a desired level of differentiation. Differentiation media provided by kits described herein may comprise any suitable components, such as those described above. For example, differentiation media may comprise one or more growth factors, such as FGF-2, FGF-8, and/or SHH. As stated above, kits may further comprise any suitable equipment for use in forming a hydrogel for implantation and/or for implanting a hydrogel into a subject. For example, kits may comprise petri dishes (e.g. for stem cell culturing), flasks, vials, beakers, syringes/needles (e.g. for loading cells into a hydrogel and/or for loading a hydrogel into a subject), centrifuges or other vortexing instruments (e.g. for vortexing hydrogels with growth factors and/or stem cells); well plates (e.g. for plating a hydrogel mixture as it “gels”), drills (e.g. for implanting a hydrogel into a subject), and any other suitable components.

### EXAMPLES

In vitro and in vivo experiments were conducted to evaluate efficacies of exemplary hydrogels for neurodegenerative disease treatment applications. In vitro data were collected to evaluate the effect of different methods of culturing on stem cell differentiation, and to assess migration potential of stem cells in a hydrogel. In vivo data were collected to evaluate the effect of two hydrogel preparations loaded with stem cells implanted into the brains of Parkinsonian mice. Hydrogels were prepared according to the following method. Lyophilized peptide powder was procured commercially comprising the sequence Fmoc-VHAVA-COOH (SEQ ID NO: 8) was mixed with 20mM sodium phosphate buffer, pH 7.4. A few drops of 2N NaOH were added to this mixture to raise the pH so that the peptide dissolved in the solution. The mixture was then sterile filtered via a 0.22 μm syringe filter in a sterile bio-safety hood. 2N HCl was gradually added to the filtered solution while vortexing to get the pH to 7.4. The mixture was vortexed while HCl was added to achieve a uniform mixture. The peptide solution was kept undisturbed at room temperature until the gelation process was complete, and the resulting hydrogel was kept at room temperature overnight. In some of the experiments described below, growth factors were added to the hydrogel after the hydrogel was formed.

### Example 1: In vitro studies

In vitro studies were conducted to evaluate the effect of different methods of stem cell culturing, and to assess the migration potential of mesenchymal stem cells in the hydrogel. Studies were also conducted to measure the loading capacity of hydrogels.

### Stem Cell Differentiation Studies

Various methods of cell culturing were evaluated to determine which method was most advantageous to achieve a desired level of stem cell differentiation. The efficacy of each method was evaluated based on tyrosine hydroxylase expression and nestin expression measured at different time points, as described below. The purpose of evaluating the cell culture protocol was to develop a method that optimized stem cell differentiation.

### Cell Preparation Methods

Frozen stem cells were revived according to the following method. Green fluorescent protein (GFP) tagged human mesenchymal stem cells (hMSCs) were revived from a frozen stock and cultured for 24 hours in complete media including serum, growth supplements, and antibiotics. Cells were cultured according to the manufacturer's protocol. A complete media was made by mixing 400 mL of DMEM base media with 50 mL of fetal bovine serum (FBS), 5 mL of L-Glutamine, and 5 mL of Penicillin-Streptomycin Solution.

The stem cells were cultured in a hydrogel exposed to differentiation media such that the media created a gradient of growth factors within the hydrogel. The differentiation media comprised neurobasal media, and growth factors B27, FGF8, and SHH (N-terminus). The hydrogel was not loaded with growth factors prior to culturing the stem cells in the hydrogel. However, the growth factors in the media diffused through the hydrogel to create a gradient of growth factors, which may aid in the terminal differentiation of the stem cells to dopamine producing cells. The cells were cultured in the hydrogel for 21 days.

### Protein Expression

Nestin expression and TH gene expression were evaluated to determine the state of differentiation of the stem cells in the hydrogel. Nestin expression was evaluated at day 14, and TH gene expression was evaluated at day 21. Images of stained stem cells within a hydrogel versus a control set of stem cells were assessed to evaluate the effects of a hydrogel on cell migration.

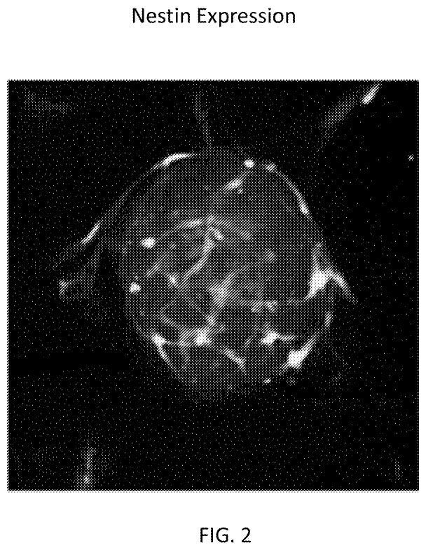

Nestin expression was evaluated at day 14 after the stem cells were loaded into the hydrogel to assess the differentiation of the stem cells. Nestin serves as an early marker that a stem cell has committed to becoming a neural cell, but has not yet matured completely. FIG. 2 depicts an image generated from immunostained cells stained for nestin. The lighter colored filaments in the image represent nestin. This image demonstrates that the stem cells cultured in the hydrogel exhibit nestin expression at day 14, suggesting that the stem cells are primed to differentiate into dopamine producing cells.

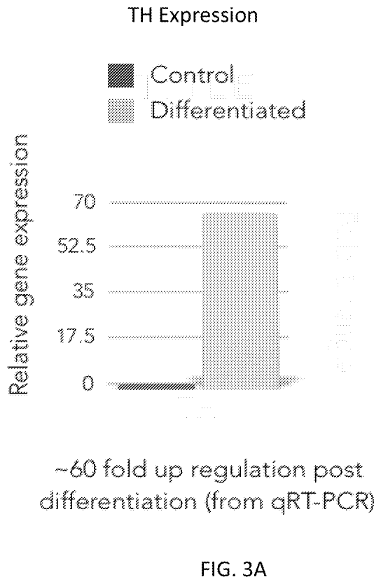

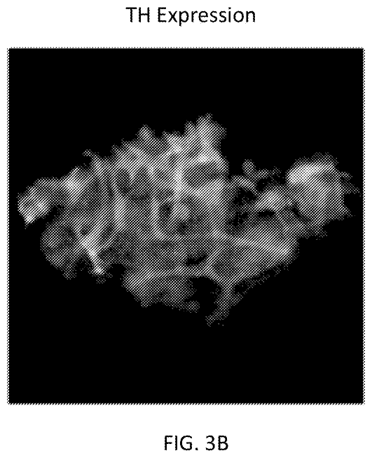

Expression of the TH gene, a marker of dopamine production, was measured at day 21 after the stem cells were loaded into the hydrogel to assess the differentiation of stem cells. To quantify TH gene expression, total RNA was extracted from the cultured cells with Trizol and quantified using Nanodrop Spectrophotometer. Total RNA was reverse transcribed to cDNA using cDNA synthesis kit (Thermo Scientific) according to manufacturer's instructions. The expression of target gene TH was quantified with SYBR green chemistry with GAPDH as the housekeeping gene HPRT, and normalized to GAPDH. Relative gene expression was determined by ΔΔCt method. Mesenchymal stem cells in the control set did not receive any differentiation cue (neither mechanical nor bio-chemical). The bar graph in FIG. 3A depicts relative gene expression of TH in the cells cultured in the hydrogel, as compared to expression of a housekeeping gene in the control condition. TH gene expression was sixty fold greater than the control, indicating increased stem cell differentiation when the cells were cultured in the hydrogel. FIG. 3B is an image generated from immunostaining for TH of an experimental hydrogel containing stem cells. TH expression is indicated by the lighter color filaments in the image. This image shows that cells are exhibiting TH expression at day 21, indicating that at least a subset of the stem cells have continued to differentiate towards fully formed dopamine producing cells.

### Stem Cell Migration

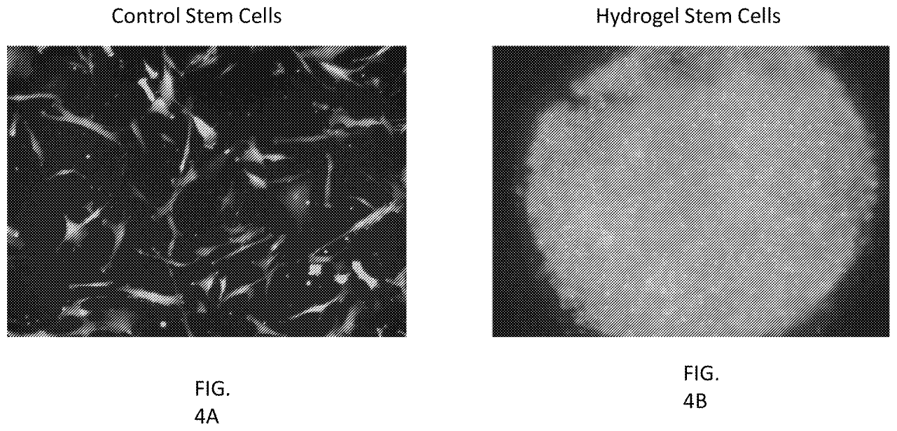

A second in vitro study was conducted to evaluate stem cell migration. Green fluorescent protein stained stem cells were prepared according to methods described above. The revived cells were split and seeded at a density of approximately 125 cells per square cm in T175 flasks. At 80% confluency the cells were further harvested and re-seeded. Media was changed every 4^{th} day. Changing complete media was preceded by a brief wash with DPBS. After a sufficient number of cells were cultured to 80% confluency with healthy morphology, cells were loaded into the hydrogel. Stem cells were loaded into the hydrogel by mixing the stem cells with the hydrogel at a density that allowed for individual cell visualization. The stem cells were loaded into the hydrogel to achieve a cell density in the range of approximately 35,000-50,000 cells per 25 μL of hydrogel. The hydrogel was evaluated to determine the confinement of hMSCs within the hydrogel compared to a control set of cells cultured on polystyrene plates. Mesenchymal stem cells in the control set did not receive any differentiation cues, neither mechanical nor bio-chemical. After 24 hours of seeding in the hydrogel, cells were set up for live-cell imaging in a Tokai Hit imaging incubator chamber. Time lapsed images were obtained for 24 hours. Stem cells in FIGS. 4A and 4B are depicted in grey. FIG. 4A depicts the control cells loaded onto polystyrene plates, and FIG. 4B depicts the cells loaded into the hydrogel. Comparing FIG. 4A to FIG. 4B, stem cells loaded into the hydrogel exhibit decreased cell migration than the cells cultured on a polystyrene plate. This suggests that the hydrogel is effective in confining the stem cells within the hydrogel matrix.

### Hydrogel Loading Density

Studies were also conducted to determine the ability of the hydrogel to support stem cells at various densities. Stem cells were seeded in hydrogels at ratios of 150,000, 200,000, 300,000, 400,000, and 500,000 cells per 20 uL of hydrogel prepared according to methods described above. For each experimental hydrogel, stem cells were mixed with 10 uL of hydrogel. A 12 mm treated coverslip was placed in a well of a 24 well-plate, 10 uL of hydrogel was placed in each well, and 10 μL of the hydrogel loaded with cells was drop cast on each 10 μL hydrogel bed. The entire system was left undisturbed for 15 minutes to allow the hydrogel to solidify. 500 μL of complete media was then added without disturbing the hydrogel, and incubated for 24 hours. Post 24 hours, the media was aspirated and fresh serum free differentiation media was added to each well. The results of this experiment indicate that stem cells seeded at a ratio of 400,000 stem cells in 20 μL of hydrogel resulted in a hydrogel loaded with stem cells that is stable over a period of 21 days.

### Example 2: In Vivo Studies

In vivo studies were conducted to evaluate the effects of two hydrogel preparations, each loaded with stem cells and implanted in Parkinsonian mice with MPTP lesioning provided according to the procedure described below. Hydrogels loaded with stem cells were implanted into Parkinsonian mice, and mice were evaluated for behaviors indicative of increased motor functions.

### Methods of Hydrogel Preparation and Implantation

Two different hydrogel preparations were tested in mice. Hydrogels loaded with stem cells were prepared according to the following method. Human mesenchymal stem cells were cultured in a T75 flask with complete media comprised of basal media, 10% FBS, 1% serum, and 1× L-Glutamine until they reached 80% confluency. Two hydrogel constructs were formed. Hydrogel construct A was formed by combining 1 mg of peptide power with 200 μL of buffer solution, NaOH, and NaCl. The peptide powder used was comprised of peptides with SEQ ID NO. 8 (Table 2). Hydrogel construct B was formed in the same manner as construct A, but was subsequently loaded with growth factors. After a hydrogel was formed, 100 ng/mL of FGF-8 and 250 ng/mL SHH N-terminus were added to the hydrogel by vortexing to form hydrogel construct B. The two formulations of hydrogel were then loaded with stem cells, and implanted into mice. To load stem cells into each hydrogel preparation, a pellet of stem cells was formed by trypsinizing cells and centrifuging at 1200 G for five minutes. The pellet was dissolved in 30 uL of each hydrogel, transiently liquefied by vortexing. In preparation A (“prep A”) 20,000 stem cells were loaded into 20 μL of hydrogel construct A. In preparation B (“prep B”), 20,000 stem cells were loaded into 20 μL of hydrogel construct B, containing 100 ng/mL of FGF-8 and 250 ng/mL SHH N-terminus. To form the suspension used for implantation, 10 μL of each construct was loaded into syringe.

For both the control and experimental hydrogel and cell mixtures, 10 μL of the hydrogel, containing approximately 20,000 cells, was loaded into a Hamiltonian needle. A burr hole was drilled into the skull of each of seven mice at a specific co-ordinate and the needle with cell formulation was inserted to the desired depth in the dorsal caudate putament (AP, −9.62; ML, 1.25 mm; DV, −2.3 mm) over a 10 minute period. The co-ordinates of cell transplant were pre-determined. Four mice received prep A, and three received prep B. The needle was fitted to the stereotactic frame and its plunger to a syringe pump and the flow rate was controlled to 1 μL/min. The injection procedure led the hydrogel to flow under the sheer force generated by pushing the piston. The formulation was inserted into the brain as a cluster of cells suspended in the hydrogel matrix in a “sol” or liquefied state. The needle was kept undisturbed for 5 minutes, so that the hydrogel reverted back to the gel state from the sol state. The needle was then withdrawn slowly and the skin over the skull was sutured.

A total of seven mice received treatment. Prior to hydrogel implantation, 12-week-old C57B16 male mice were treated with MPTP toxin to induce Parkinsonism. Four doses of 15 mg/kg free base MPTP was injected every two hours for a total of 60 mg/kg free base total in each mouse. Three weeks post-MPTP treatment, it was observed that the mice moved with reduced velocity, an indication of Parkinson's disease. Four of the mice received prep A, and three received prep B. A true control set of mice not treated with any hydrogel stem cell formulation was not kept due to the small number of animals available. Various tests were conducted to evaluate the effect of each implanted hydrogel-cell formulation.

### Behavioral Tests

Behavioral tests were performed on mice to evaluate the effects of the hydrogel on motor functioning. Behavioral tests were performed prior to MPTP, after MPTP but prior to hydrogel implantation, and at 14, 21, and 42 days post implantation (“DPI”). At each time point, one mouse was sacrificed from each group (prep A and prep B). Two behavioral tests were reported at each time point: an open maze test and a vertical rearing test. Results of the behavioral tests were compared between mice treated with prep A and mice treated with prep B.

### Open Maze

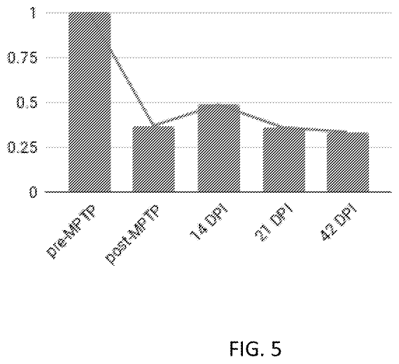

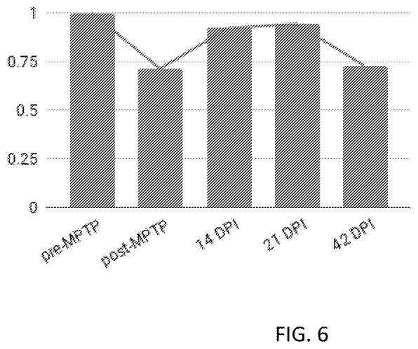

In the open maze testing, each treated mouse was placed in the center of a square area measuring 80 centimeters in diameter with a white floor. The behavior of each mouse was continuously recorded with a video camera for analysis. Between testing of each mouse, the area was cleaned with water and dried. Behavioral data was analyzed using Ethovision XT software. The arena was virtually divided into a central zone and a border zone to facilitate data analysis. The software was used to measure the velocity of each mouse, total distance travelled by each mouse, the frequency of mice entering the central zone, the duration of time spent in each zone, and the time each animal spent immobile and in locomotion. FIG. 5 depicts normalized average velocity of the mice that received prep A treatment at each time point, and demonstrates a normalized trend in recovery from PD symptoms in MPTP Parkinsonian Mice treated with prep A. FIG. 6 depicts normalized average velocity of the mice that received prep B treatment at each time point, and demonstrates a normalized trend in recovery from PD symptoms in MPTP Parkinsonian Mice treated with prep B.

### Vertical Rearing

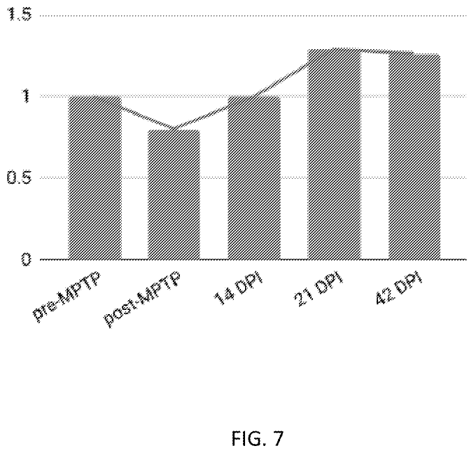

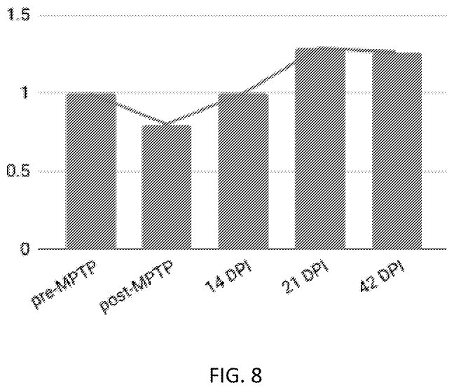

In the vertical rearing tests, each mouse was placed in a cylinder, and the number of vertical rearing movements, characterized by the mouse placing one or both forelimbs on a wall of the cylinder (“wall explorations”), were measured. Wall explorations were counted for a three minute period, and forelimb placements of right and left limbs of each mouse were observed and recorded. A rearing movement was recorded when a mouse placed a left limb, a right limb, or both limbs on the wall of the cylinder. Vertical rearing is a complex movement, and requires much more co-ordination than linear movement. Therefore vertical rearing is indicative the ability of mice to perform complex motions, demonstrating increased functioning in the brain. FIG. 7 depicts normalized average vertical rearing of mice that received prep A at each time point, and demonstrates a normalized trend in recovery from PD symptoms in MPTP Parkinsonian Mice. FIG. 8 depicts normalized average vertical rearing of mice that received prep B at each time point, and demonstrates a normalized trend in recovery from PD symptoms in MPTP Parkinsonian Mice

### Microscopy Imaging

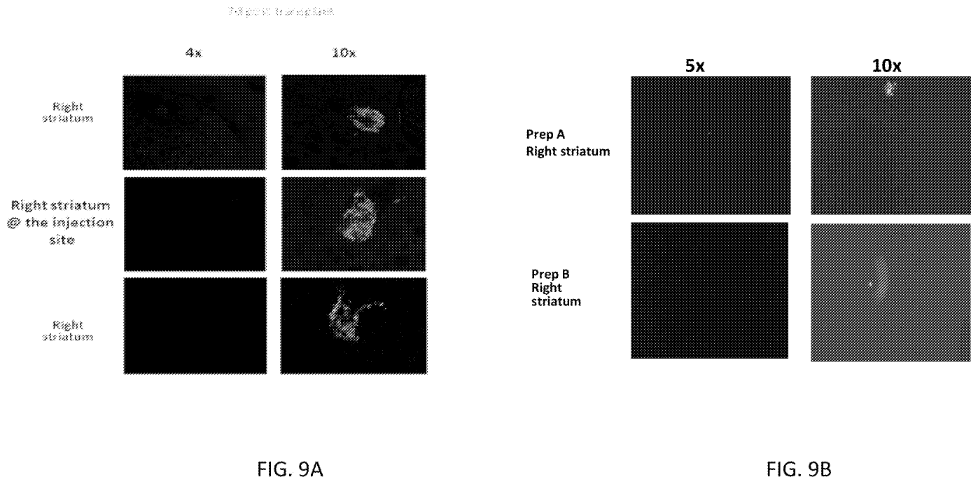

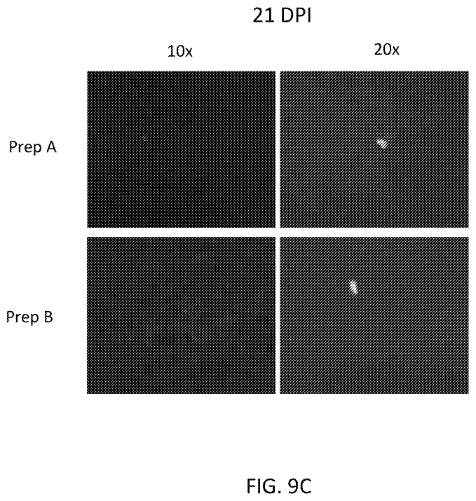

Evaluation of microscopy images of stained brain sections of mice was performed to assess the ability of the hydrogel to maintain the viability of the stem cells. One mouse was sacrificed at each time point, 7, 14, 21, and 42 DPI, and the brains were harvested and sectioned to measure the number of surviving implanted stem cells. Mice were sacrificed by cervical dislocation, and the brains were quickly removed, fixed in 4% paraformaldehyde for 2-3 days, and cryopreserved in 30% sucrose for 3 days. Brains were then cryosectioned. 30 micron thick coronal sections, out to 0.5 mm on either side of transplant site, were collected on Superfront Plus slides, and slides were frozen and stored. A subset of the coronal sections were coverslipped for microscopy and viewed under a Nikon fluorescence microscope. FIG. 9A is a series of microscopy images of brain slices with stained cells (depicted in light grey) imaged at 4× and 10× magnification at 7 DPI for prep A. At 7 DPI, GFP stained cells can be identified at the injection site at 10× magnification. FIG. 9B is a series of images of brain slices with stained cells (depicted in light grey) imaged at 5× and 10× magnification at 14 DPI for both prep A and prep B. Stained cells can be identified at 10× magnification. FIG. 9C is a series of images of brain slices with stained cells (depicted in light grey) imaged at 10× 20× magnification at 21 DPI for prep A and prep B. Stained cells can be identified at 20× magnification. These images show that the signal from the stained cells diminished overtime such that the visibility of stained cells decreased from 7 to 21 DPI.

### Example 3: Hydrogels Generated from Non-Amyloid Proteins

As an example of hydrogels based on self-aggregating peptides derived from non-amyloid proteins, laminin hydrogels were generated. Laminin was analyzed in silico to identify self-aggregating regions. After identification of such a region in the protein, its sequence was further modified to facilitate peptide self-assembly, and subsequently cross-beta sheet formation. Three peptides, L1-L3, were designed. The peptides were synthesized by a commercial vendor with 99% purity.

For forming the hydrogels, 1 mg peptide was mixed with 180 μL of 20 mM phosphate buffer, pH 7.4. The pH of this solution was raised to 10 by the gradual addition of 2N NaOH. NaCl was added to this solution in different quantities with a range from 50 mM to 150 mM final concentration. The pH of the solution was finally adjusted to 7.4 and the final volume of the solution was 200 μL. The solution was left undisturbed for 24 hours to facilitate the hydrogel formation. Within the conditions of the experiment, L1 and L3 formed hydrogels.

To determine the viability of cells within the hydrogels formed by L1 and L3, a Calcein-AM based toxicity assay was carried out. SHSY5Y cells were used for the toxicity assay. Cells were cultured in complete DMEM with 10% FBS, 1× Glutamax and 1% pen-strep antibiotic cocktail in a humidified incubator at 37° C. with 5% CO₂. Cells cultured at 80% confluency in the culture vessel were used for the assay. SHSY5Y cells were washed with phosphate-buffered saline and treated with Accutase® to lift them of the culture vessel. Subsequently, it was neutralized with a double amount of complete media and centrifuged at 300 rpm for 5 minutes to obtain a cell pellet. Around 10,000 live cells (counted on a hemocytometer by Trypan blue exclusion) were added to 20 μL of each hydrogel and plated in a well of a 96 well polystyrene TC plate (with technical replicate=3). After 20 minutes, 100 μL complete media was added to each well. For control, a similar number of cells were added to a well without the hydrogel. 20 μL of phosphate buffer was added to each of the control wells. Cells were cultured for 24 hours before performing the Calcein-AM assay. Calcein-AM assay was carried out according to manufacturer's protocol. The viable cells were imaged with a wide-field fluorescence microscope and quantified. As shown in FIG. 10, cells were 85% and 87% viable compared to control in the L1 and L3 hydrogels, respectively.

1. A composition comprising a hydrogel and modified stem cells, for implantation into a subject, wherein the hydrogel is generated from self-aggregating peptides derived from an amyloid protein, wherein the hydrogel contains one or more growth factors, and wherein the cells are modified prior to being loaded into the hydrogel.

2. The composition of claim 1, wherein the stem cells are modified by culturing in a media comprising one or more growth factors.

3. The composition of claim 1, wherein the stem cells are modified to a partially differentiated state.

4. The composition of claim 1, wherein at least a subset of the self-aggregating peptides are derived from an alpha-synuclein protein.

5. The composition of claim 1, wherein the peptides are derived from a protein that is not alpha-synuclein or beta amyloid.

6. The composition of claim 4, wherein the peptides comprise a modified non-amyloid-β component (NAC) region derived from an alpha-synuclein protein, and wherein the modified NAC region does not comprise a tyrosine amino acid.

7. The composition of any of claims 1-6, wherein the peptides further comprise an Fmoc protected N-terminus.

8. The composition of any one of claims 1-7, wherein the amino acid sequence of the peptides is selected from the sequences presented in Table 2.

9. The composition of claim 8, wherein the amino acid sequence of the peptides comprises VHAVA (SEQ ID NO: 8).

10. The composition of any of claims 1-7, wherein the peptides have a length of approximately 2-12 amino acids.

11. The composition of any of claims 1-10, wherein the hydrogel is generated from a plurality of identical self-aggregating peptides.

12. The composition of any of claims 1-10, wherein the hydrogel is generated from a mixture of different self-aggregating peptides.

13. The composition of any of claims 1-12, wherein the cells are cultured in the media until a subset of the cells reached a dopaminergic state.

14. The composition of any of claims 1-13, wherein the cells are cultured in the media until a subset of cells exhibited upregulated tyrosine hydroxylase, nestin, and/or beta-III tubulin expression.

15. The composition of any of claims 1-14, wherein the media does not comprise the growth factor FGF-2.

16. The composition of any of claims 1-15, wherein the media comprises SHH and FGF-8 growth factors.

17. The composition of any of claims 1-16, wherein the cells are cultured in the media for approximately 4-6 days.

18. The composition of any of claims 1-17, wherein the stem cells are mesenchymal stem cells.

19. The composition of any of claims 1-18, wherein the hydrogel comprises cells at a concentration within the range of approximately 200-4000 cells per μg of peptide.

20. The composition of any of claims 1-19, wherein the hydrogel has a pH in the range of 6.5-7.5.

21. The composition of any of claims 1-20, wherein the hydrogel comprises NaCl.

22. The composition of any of claims 1-20, wherein the hydrogel does not comprise NaCl.

23. The composition of any of claims 1-22, wherein the pore size of the hydrogel is between 2 and 15 μm.

24. The composition of any of claims 1-23, wherein the hydrogel is stable for 14-42 days when implanted into a subject.

25. The composition of any of claims 1-24 wherein the modified stem cells remain viable for at least 15-20, 20-15, 25-30, 30-35, or 35-40 days after the hydrogel is implanted into a subject.

26. The composition of claims 1-25, wherein the stem cells contained in the hydrogel are capable of differentiating to produce functional neurites.

27. The composition of claims 1-26, wherein the stem cells contained in the hydrogel are capable of differentiating into functional neurons.

28. A hydrogel generated from self-aggregating peptides, wherein the peptides are derived from a non-amyloid protein, wherein the protein comprises a region which when isolated forms β-sheet rich structures.

29. The hydrogel of claim 28, wherein the non-amyloid protein is laminin.

30. The hydrogel of any of claims 28-29, wherein the peptides are derived from the region of the non-amyloid protein which when isolated forms β-sheet rich structures.

31. The hydrogel of any of claims 28-30, wherein the peptides further comprise an Fmoc protected N-terminus.

32. The hydrogel of any of claims 28-31, wherein the amino acid sequence of the peptides is selected from the sequences presented in Table 3.

33. The hydrogel of claim 32, wherein the amino acid sequence of the peptide comprises SEQ ID NO: 11.

34. The hydrogel of claim 32, wherein the amino acid sequence of the peptide comprises SEQ ID NO: 13.

35. The hydrogel of any of claims 28-34, wherein the amino acid sequence of the peptide comprises SEQ ID NO: 14.

36. The hydrogel of any of claims 28-35, wherein the hydrogel forms within 24 hours of peptide incubation.

37. The hydrogel of any of claims 28-36, wherein the hydrogel comprises one or more growth factors.

38. The hydrogel of any of claims 28-37, wherein the hydrogel is loaded with modified stem cells.

39. The hydrogel of claim 38 wherein the stem cells are mesenchymal stem cells.

40. The hydrogel of any of any of claims 28-39, wherein a subset of the cells are in a dopaminergic state.

41. The hydrogel of any of any of claims 28-40, wherein a subset of the cells express nestin and/or beta-III tubulin.

42. The hydrogel of any of any of claims 28-41, wherein a subset of the cells exhibit upregulated tyrosine hydroxylase expression.

43. The hydrogel of any of claims 28-42 wherein the hydrogel is generated from a plurality of identical self-aggregating peptides.

44. The hydrogel of any of claims 28-42, wherein the hydrogel is generated from a mixture of different self-aggregating peptides.

45. The hydrogel of any of claims 28-44, wherein the hydrogel comprises cells at a concentration within the range of approximately 200-4000 cells per μg of peptide.

46. The hydrogel of any of claims 28-45, wherein the hydrogel has a pH in the range of 6.5-7.5.

47. The hydrogel of any of claims 28-46, wherein the hydrogel comprises NaCl.

48. The hydrogel of any of claims 28-46, wherein the hydrogel does not comprise NaCl.

49. The hydrogel of any of claims 28-48, wherein the pore size of the hydrogel is between approximately 2 and 15 μm.

50. A method for treating a neurological disease in subject in need thereof, comprising implanting into the subject the hydrogel of any of claims 1-49.

51. The method of claim 50, comprising implanting the hydrogel into the brain of the subject.

52. The method of any of claims 50-51, comprising implanting the hydrogel into the striatum region of the brain.

53. The method of any of claims 50-52, wherein the subject suffers from Parkinson's disease.

54. The method of any of claims 50-53, wherein the subject shows an improvement in dopamine induced locomotor movements.

55. The method of any of claims 50-54, wherein the modified stem cells of the hydrogel are viable for at least approximately 15, 20, 25, 30, 35, 40, or greater than 40 days after implantation.

56. The method of any of claims 50-55, wherein the modified stem cells of the hydrogel exhibit differentiation into functional neurons after implantation into a subject.

57. The method of claims 50-56, wherein the implanted modified stem cells decrease, ameliorate, or prevent at least one symptom of a neurological disorder.

58. A method of preparing the hydrogel of any of claims 1-49 for implantation into a subject comprising:
a. culturing stem cells in a media comprising a plurality of growth factors;
b. providing a hydrogel generated using self-aggregating peptides derived from an amyloid protein, wherein the hydrogel comprises one or more growth factors;
c. loading the stem cells into the hydrogel; and
d. implanting the hydrogel loaded with stem cells into the subject.

59. The method of claim 58, wherein the hydrogel is formed by mixing a powder-form of the self-aggregating peptides with a buffer solution.

60. The method of claim 59, comprising sterilizing the peptide powder.

61. The method of any of claims 59-60 comprising sterilizing the buffer solution.

62. The method of claim 61 wherein the sterilizing the buffer solution comprising filtering the solution through a sterile syringe filter.

63. The method of any of claims 58-62, wherein the media comprises SHH and FGF-8 growth factors.

64. The method of any of claims 58-63, wherein the media does not contain FGF-2.

65. The method of any of claims 58-64, wherein the stem cells are cultured in the media for approximately 4-6 days.

66. The method of any of claims 58-65, wherein the stem cells are mesenchymal stem cells.

67. The method of any of claims 58-66 wherein the hydrogel comprises cells at a concentration in the range of approximately 200-4000 cells per μg of peptide.

68. The method of any of claims 58-67, wherein the hydrogel has a pH in the range of 6.5-7.5.

69. The method of any of claims 58-68, wherein the hydrogel comprises NaCl.

70. The method of any of claims 58-68, wherein the hydrogel does not comprise NaCl.

71. The method of any of claims 50-70 wherein the hydrogel has a pore size in the range of approximately 2-15 μm.

72. The method of any of claims 58-71, wherein a subset of the stem cells express nestin and/or beta-III tubulin.

73. The method of any of claims 58-72, wherein a subset of the stem cells exhibit upregulated tyrosine hydroxylase expression.

74. The method of any of claims 58-73, wherein the modified stem cells of the hydrogel are viable for at least approximately 15, 20, 25, 30, 35, 40, or greater than 40 days after implantation.

75. The method of any of claims 58-74, wherein the modified stem cells of the hydrogel exhibit differentiation into functional neurons after implantation into a subject.

76. The method of any of claims 58-75, wherein the implanted modified stem cells decrease, ameliorate, or prevent at least one symptom of a neurological disorder.
